(19) United States
(12) Patent Application Publication
(10) Pub. No.: US 2010/0022975 A1
(43) Pub. Date: Jan. 28, 2010

(54) Ostomy appliance apparatus

(76) Inventor: Kalvin K. Vanden Bosch, Zeeland, MI (US)

Correspondence Address: THE WATSON INTELLECTUAL PROPERTY GROUP, PLC, 3133 HIGHLAND DRIVE, SUITE 200, HUDSONVILLE, MI, 49426, US

(21) Appl. No.: 12/380,985

(22) Filed: Mar. 5, 2009

Related U.S. Application Data
(60) Provisional application No. 61/068,195, filed on Mar. 5, 2008.

(51) Int. Cl. A61F 5/445

(52) U.S. Cl. 604/338

(57) ABSTRACT

An ostomy appliance apparatus comprising a integrally molded dome. The integrally molded dome assembly includes a dome, a drainage outlet in fluid communication with the dome, and a flange extending from the dome. An adhesive is coupled to the bottom surface of the flange. A safety disc may be supplied which extends about the outer perimeter of the flange with a portion thereof overhanging the flange.

This application claims priority from U.S. Prov. Pat. App. No. 61/068,195 filed Mar. 5, 2008, entitled “Ostomy Appliance Apparatus” and the entire disclosure is hereby incorporated by reference in its entirety.

### BACKGROUND OF THE DISCLOSURE

1. Field of the Disclosure The disclosure relates in general to a medical device, and more particularly, to an ostomy appliance apparatus which provides egress for fluids from the body of the user, so that the user can resume a normal life.

2. Background Art

The use various ostomy appliance apparatuses is well known in the art. Among other devices, the following prior art reference have been uncovered for various ostomy devices, namely, U.S. Pat. No. 6,582,410 issued to Rutman; U.S. Pat. No. 6,332,879 issued to Nielsen et al; U.S. Pat. No. 4,867,749 issued to Steer; U.S. Pat. No. 4,095,599 issued to Simonet-Haibe; U.S. Pat. No. 2,536,036 issued to Cloninger; U.S. Pat. No. 2,496,175 issued to Perry and U.S. Pat. No. 2,458,308 issued to Smoot. While each of these provide an improvement to a user by resuming a normal life, there have been drawbacks.

Thus, it is another object of the invention to overcome the deficiencies of the prior art and to provide an improved ostomy appliance apparatus.

This object as well as other objects of the present invention will become apparent in light of the present specification, claims, and drawings.

### SUMMARY OF THE DISCLOSURE

The disclosure is directed to an ostomy appliance apparatus comprising a integrally molded dome. The integrally molded dome assembly includes a dome, a drainage outlet in fluid communication with the dome, and a flange extending from the dome. An adhesive is coupled to the bottom surface of the flange.

In certain embodiments, a safety disc may be supplied which extends about the outer perimeter of the flange with a portion thereof overhanging the flange.

In a preferred embodiment, the integrally molded dome assembly includes an outside surface, an inside surface and a bottom. The flange extends from the bottom of the integrally molded dome and includes an outer portion and an inward portion. The outward portion is contained within the integrally molded dome and the outer portion extends outwardly from the bottom of the integrally molded dome.

In another embodiment, the inner portion of the flange terminates to define an opening which provides ingress into the cavity.

In yet another embodiment, the inward portion and the outward portion of the dome each have a width. The width of each of the inward and outward portions are substantially identical.

In a preferred embodiment, the width of the flange is approximately between ½″ and 1½″ and the diameter of the dome is approximately between 2″ and 4″.

In another preferred embodiment, the adhesive is positioned on each of the outward portion and the inward portion of the flange.

In yet another embodiment, the adhesive extends about substantially the entirety of the outward and inward portion of the flange.

Preferably, the flange includes an outward portion. The outward portion further includes a top surface. The safety disc is applied so as to extend over the entirety of the top surface of the outward portion of the flange.

In a preferred embodiment, the drainage outlet extends from the dome so as to be substantially parallel to the flange, to in turn, allow for positioning in, for example a downward position which is substantially parallel to a body of a user.

In another preferred embodiment, the ostomy appliance apparatus further includes a plurality of ostomy appliance apparatuses each having a dome diameter. At least two of the dome diameters of the plurality of ostomy appliance apparatuses are of different diameter so as to provide a kit.

In another preferred embodiment, the ostomy appliance apparatus further comprises a collection system including a tubular member and a collection container, the tubular member providing fluid communication between the dome and the collection container.

### BRIEF DESCRIPTION OF THE DRAWINGS

The disclosure will now be described with reference to the drawings wherein:

FIG. 1 of the drawings is a perspective view of an embodiment of the ostomy appliance apparatus of the present invention;

FIG. 2 of the drawings is a top plan view of an embodiment of the ostomy appliance apparatus of the present invention;

FIG. 3 of the drawings is a cross-sectional view of an embodiment of the ostomy appliance apparatus of the present invention, taken generally about lines 3-3 of FIG. 2; and

FIG. 4 of the drawings is a front plan view of an embodiment of the disc of the ostomy appliance apparatus of the present invention in use.

### DETAILED DESCRIPTION OF THE DISCLOSURE

While this invention is susceptible of embodiment in many different forms, there is shown in the drawings and described herein in detail a specific embodiment with the understanding that the present disclosure is to be considered as an exemplification and is not intended to be limited to the embodiment illustrated.

It will be understood that like or analogous elements and/or components, referred to herein, may be identified throughout the drawings by like reference characters. In addition, it will be understood that the drawings are merely schematic representations of the invention, and some of the components may have been distorted from actual scale for purposes of pictorial clarity.

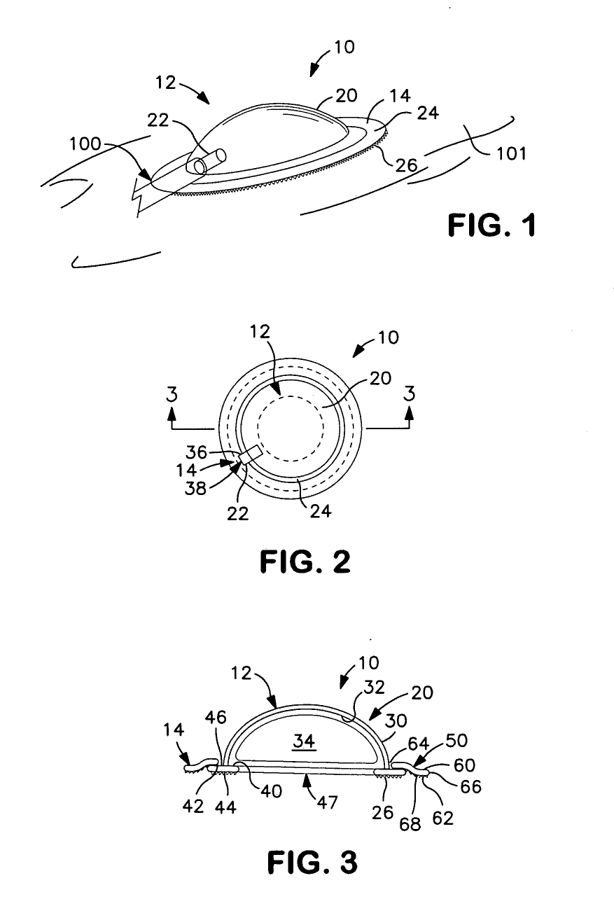

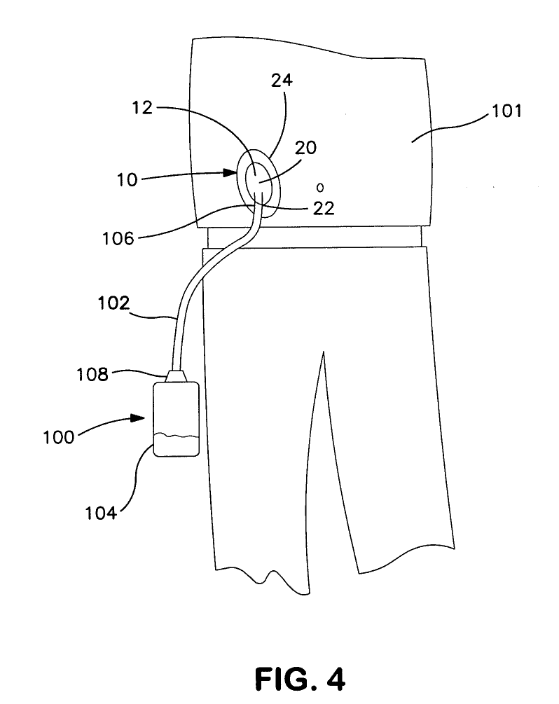

Referring now to the drawings and in particular to FIGS. 1 through 3, an ostomy appliance apparatus is shown generally at 10. The ostomy appliance apparatus of the present invention includes dome assembly 12 and safety disc 14. As is shown in FIG. 4, the apparatus is configured for attachment to a collection system 100 and to the body 101 proximate a discharge opening inserted through the abdomen. The apparatus is configured to allow for the removal of fluid from within the body and transfer thereof to the collection system.

Dome assembly 12 includes dome 20, drainage outlet 22 and flange 24. The dome assembly comprises a single integrally molded member having a flexibility to move with the body without rupturing, cracking or otherwise being compromised. It is contemplated that the dome assembly comprise a silicone member of a suitable durometer, among other synthetic polymers (such as urethanes, etc.). The dome includes outside surface 30, inside surface 32 which cooperate to define cavity 34. The drainage outlet 22 extends from the dome and includes outside surface 36 and passageway 38. The passageway is configured to be placed in fluid communication with a collection system 100, and in particular, a hose associated therewith.

The flange 24 extends from the bottom of dome 20 and includes inner portion 40, outer portion 42. The portions are separated by the dome such that the outer portion extends outside of the dome and the inner portion is within the dome. The flange further defines bottom surface 44 and top surface 46. The inner portion 40 further defines the opening 47 which provides ingress into the cavity 34.

Typically, the dome has a height of between ¾″ and 2″ and the diameter of the dome, at its largest point, is approximately 2″ to 4″. Of course, larger or smaller dome configurations are contemplated. It is likewise contemplated that the flange have a width of approximately ½″ to 1½″ wherein it may be bisected by the dome in a 50/50 relationship. Of course, other dimensions are contemplated and the invention is certainly not limited to the particular dimensions identified herein.

The dome assembly is attached to the body of the user (as is shown in FIG. 6) by way of adhesive 26 which is an adhesive that can make a fluid-tight seal between the skin of a user and the bottom surface 24 of the flange. The adhesive can be selected from a number of different adhesives that are medically approved for application upon the skin.

Additionally, a safety disc 14 may be provided which further facilitates adhesion of the apparatus to the skin. The safety disc comprises a cloth fiber (synthetic or natural) which includes a body 50 having a top surface 60, bottom surface 62, inner edge 64, outer edge 66. Adhesive 68 is applied to the bottom surface 62 of the body of the safety disc.

The safety disc is sized so that the inner edge 64 extends along the top surface of the outer portion of the flange 24 such that it may abut the outside 30 of the dome proximate the flange, or so that it may be slightly spaced apart therefrom. The outer edge 66 of the body of the safety disc 14 extends beyond the outer portion 42 of the flange of the dome assembly.

In such a configuration, the safety disc can be applied over the top surface 46 of the outer portion of the flange 24 and adhered thereto. Additionally, a portion of the adhesive 68 of the bottom surface 62 of the safety disc 14 is exposed and can be coupled to the body of the user.

In operation, and with eventual reference to FIG. 4, the user first selects the apparatus 10 that is intended for use. It will be understood that a number of embodiments can be provided, each having differently sized dome assemblies and differently shaped dome assemblies. These different sizes can be provided in order to provide accurate sizing for varying anatomies.

Once selected, the user can prepare the area of the body to which the apparatus will be attached. In particular, an alcohol swab or other cleaning fluid can be utilized to clean the immediate area surrounding the opening in the body. Next, the user can expose adhesive 26 in preparation for attachment. Typically, the adhesive 26 will be covered with a material that is typically releasable from the adhesive so as to protect the adhesive from inadvertent attachment to an outside surface.

Once this has been accomplished, the user can apply the dome assembly against the surface of the body. With the application of pressure, the adhesive bonds the bottom surface 44 of the flange to the skin of the user surrounding the opening and provides a fluid tight seal therewith. Typically, the user will attach the dome assembly with the drainage outlet 22 pointing in a downward direction so as to have the force of gravity assist drainage. Of course this orientation can be varied depending on the orientation of the user (i.e., bedridden, etc.).

In certain embodiments, it may become desirable to provide for an additional level of adhesion of the dome assembly 12 to the skin of the user. In such an instance the safety disc may be employed. In particular, the safety disc is extended over the dome (taking care to avoid the drainage outlet 22 of the dome assembly 12.

Once positioned, the safety disc 14 is pressed such that the adhesive 68 of the bottom surface 62 of the safety disc bonds to a portion of the top surface 46 of the outer portion 42 of the flange 24 and extends over the edge thereof to bond with the skin of the user immediately surrounding the flange 24. This additional safety disc provides a fluid tight seal between the flange, the skin and the adhesive of the safety disc, providing an extra measure of safety if the adhesive 26 of the flange 24 is compromised.

Once the dome assembly 12 is bonded to the body (and optionally, the safety disc is applied), collection system 100 is connected to the dome assembly. In particular, the collection system 100 generally comprises tubular member 102 and collection container 104. The tubular member 102 comprises an elongated flexible tube which, at a first end 106, is configured to matingly engage the passage 38 of the drainage outlet 22 by extending over the outside surface 36 thereof.

At a second end thereof, the tubular member 102 is coupled to a inlet 108 of the collection container 104. As a result, the cavity 34 of the dome is placed in fluid communication with the collection container 104.

The user is now able to use the system. As fluid exits the body, it is directed into the cavity 34 of the dome assembly. Next, the fluid proceeds out of the dome assembly through the drainage outlet 22. The fluid proceeds to collection system 100 and in particular, through tubular member 102 into the collection container 104. When the collection container 104 is filled it can be disconnected and replaced. Similarly, if the user wishes to replace the apparatus, the user can disconnect the adhesive from the body. A suitable replacement can then be applied.

The foregoing description merely explains and illustrates the invention and the invention is not limited thereto except insofar as the appended claims are so limited, as those skilled in the art who have the disclosure before them will be able to make modifications without departing from the scope of the invention.

What is claimed is:

1. An ostomy appliance apparatus comprising:
a integrally molded dome assembly having a dome, a drainage outlet in fluid communication with the dome, and a flange extending from the dome; and
an adhesive is coupled to the bottom surface of the flange, the adhesive structurally configured to follow the contours of an outside surface of a body of a user.

2. The ostomy appliance apparatus of claim 1 further comprising a safety disc which surrounds a portion of the flange and is coupled through adhesive thereto, a portion of the safety disc extending beyond the edge of the flange, to, in turn, facilitate adhesion to an outside surface of a body of a user.

3. The ostomy appliance apparatus of claim 1 wherein the integrally molded dome assembly includes an outside surface, an inside surface and a bottom, and the flange extends from the bottom of the integrally molded dome and includes an outer portion and an inward portion, the outward portion is contained within the integrally molded dome and the outer portion extends outwardly from the bottom of the integrally molded dome.

4. The ostomy appliance apparatus of claim 3 wherein the inner portion of the flange terminates to define an opening which provides ingress into the cavity.

5. The ostomy appliance apparatus of claim 4 wherein the inward portion and the outward portion of the dome each have a width, and wherein the width of each of the inward and outward portions are substantially identical.

6. The ostomy appliance apparatus of claim 4 wherein the width of the flange is approximately between 2″ and 1½″ and wherein the diameter of the dome is approximately between 2″ and 4″.

7. The ostomy appliance apparatus of claim 3 wherein the adhesive is positioned on each of the outward portion and the inward portion of the flange.

8. The ostomy appliance apparatus of claim 3 wherein the adhesive extends about substantially the entirety of the outward and inward portion of the flange.

9. The ostomy appliance apparatus of claim 2 wherein the flange includes an outward portion, the outward portion further includes a top surface, the safety disc is applied so as to extend over the entirety of the top surface of the outward portion of the flange.

10. The ostomy appliance apparatus of claim 1 wherein the drainage outlet extends from the dome so as to be substantially parallel to the flange, to in turn, allow for positioning in, for example a downward position which is substantially parallel to a body of a user.

11. The ostomy appliance apparatus of claim 1 further comprising a plurality of ostomy appliance apparatuses each having a dome diameter, wherein at least two of the dome diameters of the plurality of ostomy appliance apparatuses are of different diameter so as to provide a kit.

12. The ostomy appliance apparatus of claim 1 further comprising a collection system including a tubular member and a collection container, the tubular member providing fluid communication between the dome and the collection container.
